(19) United States
(12) Patent Application Publication
(10) Pub. No.: US 2021/0220245 A1
(43) Pub. Date: Jul. 22, 2021

(54) AN OILY COMPOSITION

(71) Applicant: WACKER CHEMIE AG, Munich (DE)

(72) Inventors: XINYU (STANLEY) LIANG, Shanghai (CN); YUWEN (LAURA) LIANG, Shanghai (CN)

(73) Assignee: WACKER CHEMIE AG, Munich (DE)

(21) Appl. No.: 17/268,281

(22) PCT Filed: Aug. 16, 2018
(86) PCT No.: PCT/CN2018/100788

§ 371 Date: Feb. 12, 2021

(51) Int. Cl. A61K 8/58; A61K 8/31; A61Q 19/00

(52) U.S. Cl. CPC A61K 8/585; A61K 8/31; A61K 2800/30; A61K 2800/5922; A61K 2800/10; A61Q 19/00

(57) ABSTRACT

Provided are an oily composition and use thereof. The oily composition comprises: a) a first volatile acyclic silicone oil selected from among acyclic silicone oils with 2 to 4 silicon atoms, optionally containing C1-C6 alkyl groups, and mixtures thereof; b) a second volatile acyclic silicone oil selected from among acyclic silicone oils with 5 to 6 silicon atoms, optionally containing C1-C6 alkyl groups, and mixtures thereof.

### FIELD OF THE INVENTION

The disclosure relates to an oily composition that can be used as an alternative to decamethylcyclopentasiloxane (D5) in personal care and cosmetic products or as a carrier liquid for siloxane polymers.

### BACKGROUND OF THE INVENTION

D5 is widely used in a variety of personal care and cosmetic products due to its low cost, excellent compatibility, good chemical stability, relatively uniform evaporation rate, and ability to stay on skin or hair for a certain period of time, while leaving no residues after evaporation to provide a refreshing, non-greasy conditioning effect. However, being bioaccumulative and environmentally persistent, it has negative impacts especially on aquatic organisms. Moreover, because of its specialty of production, D5 inevitably contains a certain proportion of octamethylcyclotetrasiloxane (D4), which has proved to be reproductive toxicity.

In recent years, cosmetic-related regulations on D5 have become increasingly stringent. In the Commission Regulation (EU) 2018/35 of January, 2018 amending Annex XVII to Regulation (EC) concerning the Registration, Evaluation, Authorisation and Restriction of Chemicals (REACH), both D4 and D5 have been added to Annex XVII restricted substances list (entry 70). According to the regulation, they can no longer be placed on the market in wash-off cosmetic products in a concentration equal to or greater than 0.1% by weight of either substance after Jan. 31, 2020. Due to their significant toxicity persistence and bioaccumulation, D4 and D5 were added to the Candidate List of Substances of Very High Concern (SVHC) on Jun. 27, 2018, as approved by the Member State Committee (MSC).

Therefore, it has become very important to seek a satisfactory alternative to D5 with similar volatility characteristics and comparable behavior in personal care and cosmetic product formulations particularly offering equivalent sensorial properties. So far, this topic has been involved in some prior arts. US20040241200A discloses a hydrocarbon composition to replace D5, which comprises 65% neopentyl glycol diheptanoate and 35% isododecane. WO2008155059A discloses a hydrocarbon composition as an alternative to D5, which comprises 65-70% n-undecane and 24-30% n-tridecane. EP2417199A discloses a hydrocarbon composition as a substitute for D5, which comprises 90-99% dodecane and 1-10% tetradecane. U.S. Pat. No. 8,506,974B discloses a hydrocarbon composition as a replacement for D5, which comprises 35% C12-C14 isoparaffins, 55% C13-C16 isoparaffins and 10% C13-C15 alkanes.

Although the above prior arts disclose these hydrocarbon compositions with volatility characteristics, they do not disclose specific evaporation rates, or only disclose the amount of residual compositions after a long period of time, such as 5 hours. Furthermore, all of these hydrocarbon compositions fail to offer a soft silky feel to formulations, as is the case with D5. Therefore, there is still a need for an alternative to D5 that has approximate volatility characteristics and offers very similar sensorial properties.

### SUMMARY OF THE INVENTION

The disclosure provides an oily composition that approximates D5 in terms of volatility characteristics, i.e., it can volatilize slowly at a relatively uniform rate, with 65-95 wt % or even 80-95 wt % volatilized after 2 hours measured according to DIN 53249. It also conveys similar soft silky, non-greasy sensorial properties.

As used herein, the term “oily composition” means a substance formed essentially, or even consisting, of oils, wherein “essentially” means that the oils are present in the composition in an amount of more than 80 wt %, such as more than 90 wt %, more than 95 wt %, or even more than 99 wt %.

The term “volatile” herein means at least 5 wt % of a liquid substance being capable of transforming into a vapor on contact with skin or hair in less than 2 hours, at room temperature and atmospheric pressure, wherein “liquid substance” means a substance capable of flowing under its own weight in less than 5 minutes, at room temperature.

The term “silicone oil” means a siloxane or polysiloxane that remains in a liquid state at room temperature.

The term “acyclic silicone oil” means a silicone oil that does not have a cyclic structure, including linear silicone oils and branched silicone oils.

The term “hydrocarbon-based oil” herein means an oil formed essentially, or even consisting, of carbon and hydrogen atoms, and possibly oxygen, nitrogen and fluorine atoms, and containing no silicon atoms, wherein “essentially” means that carbon and hydrogen are present in the hydrocarbon-based oil in an amount of more than 80 wt %, such as more than 90 wt %, more than 95 wt %, or even more than 99 wt %.

As used herein, “room temperature” means 23±2° C., unless otherwise specified. As used herein, “kinematic viscosity” can be measured at 25° C. according to the methods conventionally used in the art, e.g. DIN 51562.

As used herein, “flash point” can be measured according to the methods conventionally used in the art, e.g. DIN 51755.

The first aspect of the disclosure provides an oily composition comprising:

a) a first volatile acyclic silicone oil selected from among acyclic silicone oils with 2 to 4 silicon atoms, optionally containing C1-C6, preferably C1-C2 alkyl groups, and mixtures thereof;

b) a second volatile acyclic silicone oil selected from among acyclic silicone oils with 5 to 6 silicon atoms, optionally containing C1-C6, preferably C1-C2 alkyl groups, and mixtures thereof.

Ingredient (a)

Ingredient (a) typically includes volatile linear silicone oils having the following general formula I and volatile branched silicone oils having the general formula II:

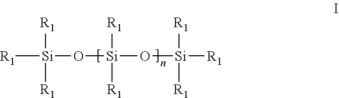

where n is an arbitrary integer between 0 and 4;

R₁ can be the same or different C1-C6 alkyl groups, such as methyl, ethyl, propyl, butyl, pentyl, hexyl, preferably methyl or ethyl, particularly preferably methyl;

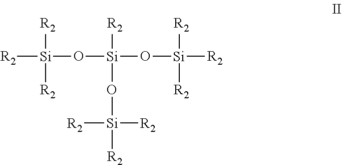

where R₂ can be the same or different C1-C6 alkyl groups, such as methyl, ethyl, propyl, butyl, pentyl, hexyl, preferably methyl or ethyl, particularly preferably methyl.

Ingredient (a) has a suitable kinematic viscosity of from 0.65 to 1.5 cSt, such as 0.65 cSt, 1 cSt or 1.5 cSt.

Ingredient (a) is suitably selected from among acyclic silicone oils with a flash point greater than or equal to −6° C. and less than 70° C., preferably linear silicone oils with a flash point of 20-40° C. or branched silicone oils with that of 50-70° C.

Non-limiting examples of ingredient (a) that may especially be mentioned include hexamethyldisiloxane, octamethyltrisiloxane, heptamethylhexyltrisiloxane, decamethyltetrasiloxane, methyl trimethicone and mixtures thereof.

Ingredient (b)

Ingredient (b) has a suitable kinematic viscosity of greater than 1.5 cSt and less than 3 cSt, such as 1.8 cSt or 2 cSt.

Ingredient (b) is suitably selected from among acyclic silicone oils with a flash point greater than or equal to 70° C. and less than 100° C., preferably linear silicone oils with a flash point of 70-95° C.

Non-limiting examples of ingredient (b) that may especially be mentioned include dodecamethylpentasiloxane, tetradecyl hexasiloxane, and mixtures thereof.

In the oily composition according to the first aspect of the disclosure, ingredients (a) can be present in an amount of from 20% to 80%, for example, 20%, 30%, 40%, 50%, 60%, 70% or 80%, ingredients (b) can be present in an amount of from 20% to 80%, for example, 20%, 30%, 40%, 50%, 60%, 70% or 80%, based on the total weight of the oily composition.

The suitable mass ratio of ingredient (a) to ingredient (b) ranges from 20:80 to 80:20, such as 20:80, 30:70, 40:60, 70:30 and 80:20. In certain embodiments, the mass ratio of ingredient (a) to ingredient (b) ranges from 70:30 to 80:20. In certain embodiments, the mass ratio of ingredient (a) to ingredient (b) ranges from 20:80 to 40:60.

Preferably, ingredients (a) and (b) are present in an amount of more than 80%, such as more than 90% or 95%, or even more than 99%, based on the total weight of the oily composition.

The second aspect of the disclosure also provides an oily composition comprising:

ingredient (a) according to the first aspect of the disclosure;

ingredient (b) according to the first aspect of the disclosure; and

c) a volatile hydrocarbon-based oil.

Ingredient (c)

Ingredient (c) is suitably selected from among hydrocarbon-based oils with a flash point of 40-110° C., preferably 40-70° C., particularly preferably 40-55° C.

Non-limiting examples of ingredient (c) that may especially be mentioned include volatile hydrocarbon-based oils containing 8 to 16 carbon atoms and mixtures thereof, in particular branched alkanes containing 8 to 16 carbon atoms or mixtures thereof, including C8-C16 isoparaffins, such as isomeric decane, isododecane and isohexadecane, particularly preferably isododecane.

According to the second aspect of the disclosure, the oily composition comprises ingredient (a) in an amount of from 10% to 40%, for example, 10%, 15%, 20%, 25%, 30%, 35% or 40%, ingredient (b) in an amount of from 20% to 40%, for example, 20%, 25%, 30%, 35% or 40%, and ingredient (c) in an amount of from 20% to 70%, for example, 20%, 30%, 40%, 50%, 60% or 70%, preferably 30-60%, based on the total weight of the oily composition.

Preferably, ingredients (a), (b) and (c) are present in an amount of more than 80%, such as more than 90% or 95%, or even more than 99%, based on the total weight of the oily composition.

According to the second aspect of the disclosure, the proportions of ingredients (a), (b) and (c) can be adjusted based on their individual volatility characteristics to obtain an overall volatility very close to D5.

In certain embodiments, the oily composition comprises octamethyltrisiloxane, dodecamethylpentasiloxane and isododecane, which, in terms of content, have the relationship: isododecane>dodecamethylpentasiloxane>octamethyltrisiloxane.

In certain embodiments, the oily composition comprises hexamethyldisiloxane, dodecamethylpentasiloxane and isododecane, which, in terms of their contents, have the relationship: isododecane>dodecamethylpentasiloxane>hexamethyldisiloxane.

In certain embodiments, the oily composition comprises decamethyltetrasiloxane, dodecamethylpentasiloxane and isododecane, which, in terms of their contents, have the relationship: decamethyltetrasiloxane>isododecane dodecamethylpentasiloxane.

According to the first or second aspect of the disclosure, the oily composition may further comprise other optional ingredients as needed, such as a suitable amount of additives, for example stabilizers, antioxidants and preservatives, provided that they do not affect the realization of the overall object of the disclosure.

Preferably, the oily composition is substantially free of one or more substances among

i) acyclic silicone oils containing more than 7 silicon atoms, including volatile and non-volatile linear or branched silicone oils;

ii) cyclic silicone oils, typically including D4, D5, D6 or derivatives thereof;

iii) water;

iv) powders, including pigments and fillers, wherein non-limiting examples of typical pigments include titanium dioxide, zinc oxide, iron oxide, chromium oxide, zirconium oxide, cerium oxide, cochineal and pearlescent particles, and non-limiting examples of fillers include talc, mica, silica, kaolin, calcium carbonate and magnesium carbonate;

v) plant-derived ingredients, including ingredients extracted from flowers, leaves, stems, roots or fruits of plants; and

vi) non-volatile oils.

The expression “substantially free” means that the content of any of the above substances i)-vi) is less than 1 wt %, such as less than 0.9 wt %, 0.8 wt %, 0.7 wt %, 0.6 wt %, 0.5 wt %, 0.4 wt %, 0.3 wt %, 0.2 wt % or 0.1 wt %, in particular less than 0.05 wt % or 0.01 wt %, based on the total weight of the oily composition.

The third aspect of the disclosure provides use of the oily composition of the first or second aspect in personal care and cosmetic products, including skin care products, hair care products, cosmetics, sunscreen products, antiperspirant products, shaving care products and nail care products, including cleansers, lotions, creams, shower gels, shampoos, conditioners, color cosmetics, antiperspirants, deodorants and nail varnishes.

The fourth aspect of the disclosure provides a personal care or cosmetic composition comprising the oily composition of the first or second aspect of the disclosure.

The suitable amount of the oily composition can be selected generally from 0.01-99%, preferably 0.5-70%, especially 1-60%, based on the total weight of the personal care or cosmetic composition, according to the desired sensations and the dosage form of the personal care or cosmetic composition, such as lotions, creams, sprays or ointments.

The fifth aspect of the disclosure also provides use of the oily composition of the first or second aspect as a carrier liquid for siloxane polymers such as silicone elastomers and polysiloxanols.

The oily composition is generally used in an amount of 0.01-98 wt %, such as 0.5-90 wt % or 5-80 wt %, based on the total weight of the siloxane polymer and the oily composition.

### BRIEF DESCRIPTION OF THE DRAWINGS

FIG. 1 is a graph depicting the evaporation curves of the oily compositions of Examples 1-3 and Comparative Examples 1-2, with D5, BELSIL® DM 1 PLUS, BELSIL® DM 2 and IP 12 used as references.

FIG. 2 is a graph depicting the evaporation curves of the oily compositions of Examples 4-8 and Comparative Examples 3-4, with D5, BELSIL® DM 1 PLUS, BELSIL® DM 2 and IP 12 used as references.

FIG. 3 is a graph showing the application of samples on a forearm for skin feel test.

### DETAILED DESCRIPTION OF THE PREFERRED EMBODIMENTS

The invention is further illustrated by the following examples, but is not limited to the scope thereof. Any experimental methods with no conditions specified in the following examples are selected according to the conventional methods and conditions, or product specifications.

The ingredients used in the Examples and Comparative Examples are all commercially available, with detailed information as follows:

BELSIL® DM 0.65 from Wacker Chemicals, a hexamethyldisiloxane, being a first volatile acyclic silicone oil with a kinematic viscosity of about 0.65 cSt measured at 25° C. according to DIN 53018, and a flash point of −6° C.;

BELSIL® DM 1 PLUS from Wacker Chemicals, a polydimethylsiloxane, being a first volatile acyclic silicone oil with a kinematic viscosity of about 1.23 cSt measured at 20° C. according to DIN 51562, and a flash point of 32° C.;

BELSIL® DM 2 from Wacker Chemicals, a polydimethylsiloxane, being a second volatile acyclic silicone oil with a kinematic viscosity of 2 cSt measured at 25° C. according to DIN 51562, and a flash point of 89° C.;

BELSIL® DM 5 from Wacker Chemicals, a polydimethylsiloxane, with a kinematic viscosity of about 5 cSt measured at 25° C. according to DIN 53019, and a flash point of 130° C.;

XIAMETER™ PMX-200 Silicone Fluid 1.5 cSt from Dow Corning, a polydimethylsiloxane, being a first volatile acyclic silicone oil with a kinematic viscosity of 1.5 cSt at 25° C. and a flash point of 57° C.;

Silsoft 034 from Momentive Performance Materials, a Caprylyl Methicone with a kinematic viscosity of 3 cSt at 25° C. and a flash point of 110° C.;

IP 12 from Idemitsu Kosan, an isododecane.

Unless otherwise specified, wt % hereinafter is based on the total weight of the oily composition.

Examples 1-3 and Comparative Examples 1-2: Preparation of oily compositions comprising volatile acyclic silicone oils and volatile hydrocarbon-based oils

The oily compositions of Examples 1-3 and Comparative Examples 1-2 were obtained by uniformly mixing all ingredients according to Table 1.

TABLE 1

| Ingredients (wt %) | Ex.1 | Ex.2 | Ex.3 | C.Ex.1 | C.Ex.2 |
|---|---|---|---|---|---|
| BELSIL ® DM 0.65 | / | 10 | / | 10 | / |
| BELSIL ® DM 1 PLUS | 10 | / | / | / | 10 |
| XIAMETER ™ PMX-200 | / | / | 40 | / | / |
| Silicone Fluid 1.5 cSt | | | | | |
| BELSIL ® DM 2 | 30 | 30 | 30 | / | / |
| Silsoft 034 | / | / | / | 30 | 30 |
| IP 12 | 60 | 60 | 30 | 60 | 60 |

Examples 4-8 and Comparative Examples 3-4: Preparation of oily compositions comprising only volatile acyclic silicone oils

The oily compositions of Examples 4-8 and Comparative Examples 3-4 were obtained by uniformly mixing all ingredients according to Table 3.

TABLE 2

| Ingredients (wt %) | Ex.4 | Ex.5 | Ex.6 | Ex.7 | Ex.8 | C.Ex.3 | C.Ex.4 |
|---|---|---|---|---|---|---|---|
| BELSIL ® | 30 | 40 | 70 | 80 | 20 | 10 | 10 |
| DM 1 PLUS | | | | | | | |
| BELSIL ® DM 2 | 70 | 60 | 30 | 20 | 80 | 90 | 80 |
| BELSIL ® DM 5 | / | / | / | / | / | / | 10 |

### Example 9: Evaporation Rate Test

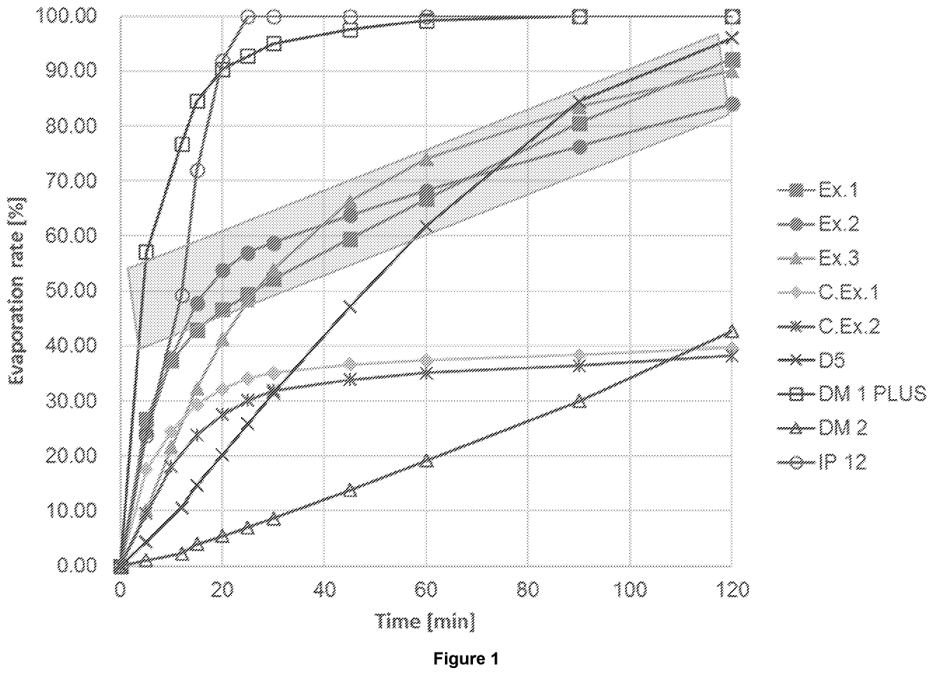

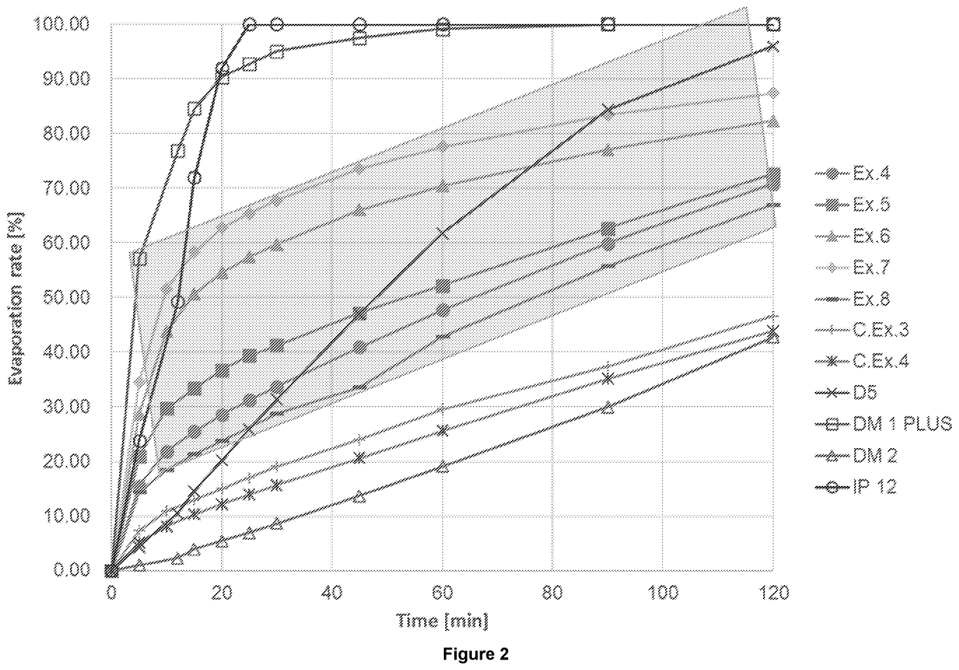

The evaporation rate curves of the oily compositions of all examples and comparative examples were tested according to DIN 53249 at 25° C., with D5, BELSIL® DM 1 PLUS, BELSIL® DM 2 and IP 12 used as references. The specific test results are shown in FIGS. 1-2, in which the ordinate indicates the percentage of the weight loss of the sample due to evaporation to the total weight of the initial sample.

It can be seen from FIG. 1 that the evaporation curves of BELSIL® DM 1 PLUS, BELSIL® DM 2 and IP 12 distinctly deviate from that of D5; the evaporation curves of the oily compositions of Comparative Examples 1-2 are closer to that of D5 than the above single substances, but still have a large deviation from that of D5; however, the evaporation curves of the oily compositions of Examples 1-3 are very close to that of D5, indicating that they can volatilize slowly at a relatively uniform rate, with 80-95 wt % volatilized after 2 hours.

As can be seen by comparing FIG. 1 with FIG. 2, the evaporation curves of the oily compositions of Examples 4-8 are closer to that of D5 than BELSIL® DM 1 PLUS, BELSIL® DM 2 and IP 12, but slightly farther from the D5 curve than Examples 1-3, with only 65-85 wt % volatilized after 2 hours. The evaporation curves of Comparative Examples 3-4 deviate from that of D5, mainly due to the high content of BELSIL® DM 2 or the inclusion of BELSIL® DM 5.

### Example 10: Cream Tests on Skin

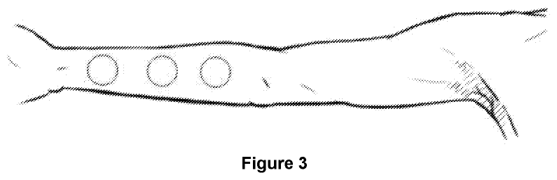

Four different skin creams A, B, C and D were prepared from D5, the oily compositions of Examples 1 & 4 and Caprylyl Methicone respectively, and evaluated by 35 panelists in terms of the skin feels in the initial, middle and late stages after the creams were applied on their forearms. The skin feels in the initial, middle and late stages after application herein means the feels on skin after 0.25 μL of the test creams were respectively applied in a circular motion on a clean and dry forearm within a circular area of 4 cm in diameter for 5 cycles, 30 cycles and until completely absorbed (see FIG. 3).

The scoring rules are as follows: using skin cream A as a reference, creams B, C and D are respectively compared with A; if A and B (or C, or D) are similar in terms of the skin feels, then each scores 0.5; if A has better skin feels than B (or C, or D), then A scores 1, and B (or C, or D) scores 0, and the final result is the average of the scores. If the scores of A and B (or C, or D) are close, then they give the similar feels on skin, while a larger score difference means a greater gap in the skin feels.

The formulations of the skin creams are shown in Table 3 below (in parts by weight).

TABLE 3

| P | | | | | | | |
|---|---|---|---|---|---|---|---|
| h | | | | | | | |
| a | | | | | | | |
| s | | Trade | | | | | |
| e | INCI Names | names | Suppliers | A | B | C | D |
| I | Cyclopentasiloxane | BELSIL ® | WACKER | 8.00 | / | / | / |
| | | CM 040 CN | | | | | |
| | Oily composition of | N/A | N/A | / | 8.00 | / | / |
| | Ex. 1 | | | | | | |
| | Oily composition of | N/A | N/A | / | / | 8.00 | / |
| | Ex. 4 | | | | | | |
| | Caprylyl Methicone | Silsoft 034 | Momentive | / | / | / | 8.00 |
| | Dimethicone, | BELSIL ® | WACKER | 3.00 | 3.00 | 3.00 | 3.00 |
| | Dimethiconol | GB 1020 | | | | | |
| | Silica Dimethyl | HDK ® H | WACKER | 1.00 | 1.00 | 1.00 | 1.00 |
| | Silylate | 20 | | | | | |
| | Ethylhexyl | Parsol ® | DSM | 5.00 | 5.00 | 5.00 | 5.00 |
| | methoxycinnamate | MCX | | | | | |
| | Caprylic/capric | CRODAMOL | CRODA | 5.00 | 5.00 | 5.00 | 5.00 |
| | triglyceride | TM GTCC | | | | | |
| | Dimethicone, | BELSIL ® | WACKER | 5.00 | 5.00 | 5.00 | 5.00 |
| | caprylyl dimethicone | WO 5000 | | | | | |
| | ethoxy glucoside | | | | | | |
| | Cetyl PEG/PPG-10/1 | ABIL ® EM | Evonik | 1.60 | 1.60 | 1.60 | 1.60 |
| | Dimethicone | 90 | | | | | |
| II | Glycerin | N/A | Emery | 3.00 | 3.00 | 3.00 | 3.00 |
| | | | Oleo- | | | | |
| | | | chemicals | | | | |
| | Butanediol | N/A | Celanese | 7.00 | 7.00 | 7.00 | 7.00 |
| | NaCI | N/A | N/A | 0.50 | 0.50 | 0.50 | 0.50 |
| | Disodium EDTA | N/A | Akzo Nobel | 0.05 | 0.05 | 0.05 | 0.05 |
| | Deionized water | N/A | N/A | Added | Added | Added | Added |
| | | | | up to | up to | up to | up to |
| | | | | 100 | 100 | 100 | 100 |
| III | Preservative | N/A | N/A | Appro- | Appro- | Appro- | Appro- |
| | | | | priate | priate | priate | priate |
| | | | | amount | amount | amount | amount |

The preparation process was as follows:

1) all ingredients of phases I and II were separately mixed well according to Table 3;

2) phases I and II were separately heated to 55° C., and then phase I was slowly added into phase II while stirring followed by homogenizing for 3-5 minutes;

3) phase Ill was added to the mixture of phase I and II to mix well.

Tables 4-6 respectively show the scores of the skin feels of creams B, C and D compared with cream A. As can be seen from Tables 4 and 5, the oily composition of Example 1 is the same as D5 in terms of almost all skin feels, except spreadability in the initial stage and silkiness in the middle stage after application; the oily composition of Example 4 is inferior to Example 1 in approximating the skin feels of D5. Table 6 shows that Caprylyl Methicone is rather different from D5 in terms of all skin feel indicators.

TABLE 4

| | | Score | |
|---|---|---|---|
| Stage | Indicator | A | B |
| Initial stage after | Spreadability | 0.2 | 0.8 |
| application | | | |
| Middle stage | Silkiness | 0.4 | 0.6 |
| after application | | | |
| Late stage after | Silkiness | 0.5 | 0.5 |
| application | Stickiness | 0.5 | 0.5 |
| | Moisture | 0.5 | 0.5 |

TABLE 5

| | | Score | |
|---|---|---|---|
| Stage | Indicator | A | C |
| Initial stage after | Spreadability | 0.2 | 0.8 |
| application | | | |
| Middle stage | Silkiness | 0.3 | 0.7 |
| after application | | | |
| Late stage after | Silkiness | 0.6 | 0.4 |
| application | Stickiness | 0.4 | 0.6 |
| | Moisture | 0.4 | 0.6 |

TABLE 6

| | | Score | |
|---|---|---|---|
| Stage | Indicator | A | D |
| Initial stage after | Spreadability | 0.1 | 0.9 |
| application | | | |
| Middle stage | Silkiness | 0.3 | 0.7 |
| after application | | | |
| Late stage after | Silkiness | 0.6 | 0.4 |
| application | Stickiness | 1 | 0 |
| | Moisture | 0.8 | 0.2 |

### Example 11: Liquid Foundation Tests on Skin

Three different liquid foundations E, F and G were prepared from D5, the oily composition of Example 1 and Caprylyl Methicone respectively, and evaluated by 35 panelists in terms of the skin feels in the initial, middle and late stages after the foundations were applied on their forearms. The skin feels in the initial, middle and late stages are as defined in Example 10. The scoring rules are as follows: using liquid foundation E as a reference, F and G are respectively compared with E; if E and F (or G) are similar in terms of the skin feels, then each scores 0.5; if E has a better skin feels than F (or G), then E scores 1, and F (or G) scores 0, and the final result is the average of the scores. If the scores of E and F (or G) are close, then they give the similar feels on skin, while a larger score difference means a greater gap in the skin feels.

The formulations of the liquid foundations are shown in Table 7 below (in parts by weight).

TABLE 7

| P | | | | | | |
|---|---|---|---|---|---|---|
| h | | | | | | |
| a | | | | | | |
| s | | | | | | |
| e | INCI names | Trade names | Suppliers | E | F | G |
| I | PEG-10 Dimethicone | KF-6017 | Shin-Etsu | 1.50 | 1.50 | 1.50 |
| | | | Chemical | | | |
| | Dimethicone, caprylyl | BELSIL ® | WACKER | 0.98 | 0.98 | 0.98 |
| | dimethicone ethoxy | WO 5000 | | | | |
| | glucoside | | | | | |
| | Polyglyceryl-3 | Lameform ® | BASF | 3.60 | 3.60 | 3.60 |
| | diisostearate | TGI | | | | |
| | Cyclopentasiloxane | BELSIL ® | WACKER | 15.91 | / | / |
| | | CM 040 ON | | | | |
| | Oily composition of Ex. 1 | N/A | N/A | / | 15.91 | / |
| | Caprylyl Methicone | Silsoft 034 | Momentive | / | / | 15.91 |
| | Dimethicone, | BELSIL ® | WACKER | 7.00 | 7.00 | 7.00 |
| | dimethicone/vinyl | REG 1102 | | | | |
| | trimethylsiloxysilicate | | | | | |
| | crosspolymer | | | | | |
| | Isododecane | IP 12 | Idemitsu | 3.00 | 3.00 | 3.00 |
| | Mineral oil | N/A | N/A | 2.00 | 2.00 | 2.00 |
| | Titanium dioxide, | TiO₂ CR-50 | K.S Pearl | 7.00 | 7.00 | 7.00 |
| | triethoxycaprylylsilane | AS | | | | |
| | Ferric oxide yellow, talc, | I.O. Yellow | K.S Pearl | 0.36 | 0.36 | 0.36 |
| | triethoxycaprylylsilane | AS | | | | |
| | Ferric oxide red, talc, | I.O. Red AS | K.S Pearl | 0.12 | 0.12 | 0.12 |
| | triethoxycaprylylsilane | | | | | |
| | Ferric oxide black, talc, | I.O. Black AS | K.S Pearl | 0.08 | 0.08 | 0.08 |
| | triethoxycaprylylsilane | | | | | |
| | Disteardimonium | BENTONE ® | Elements | 2.00 | 2.00 | 2.00 |
| | hectorite | 38 | | | | |
| II | Glycerin | N/A | N/A | 8.56 | 8.56 | 8.56 |
| | Propylene glycol | N/A | N/A | 4.28 | 4.28 | 4.28 |
| | NaCl | N/A | N/A | 0.86 | 0.86 | 0.86 |
| | Deionized water | N/A | N/A | Added | Added | Added |
| | | | | up to 100 | up to 100 | up to 100 |
| III | Preservative | N/A | N/A | Appro- | Appro- | Appro- |
| | | | | priate | priate | priate |
| | | | | amount | amount | amount |

The preparation process was as follows:

1) according to Table 7, the ingredients of phase I were mixed well except the volatile ones to form a homogeneous color paste, then the volatile ingredients were added to mix well;

2) the well-mixed phase II was slowly added into phase I while stirring followed by homogenizing for 3-5 minutes;

3) phase Ill was added to the mixture of phase I and II to mix well.

Tables 8-9 respectively show the scores of the skin feels of liquid foundations F and G compared with liquid foundation E. Tables 8-9 show that the oily composition of Ex. 1 is similar with D5 in terms of almost all skin feels, while Caprylyl Methicone is rather different from D5 in terms of all skin feel indicators.

TABLE 8

| | | Score | |
|---|---|---|---|
| Stage | Indicator | E | F |
| Initial stage after | Spreadability | 0.6 | 0.4 |
| application | | | |
| Middle stage | Silkiness | 0.7 | 0.3 |
| after application | | | |
| Late stage after | Silkiness | 0.6 | 0.4 |
| application | Stickiness | 0.5 | 0.5 |
| | Moisture | 0.7 | 0.3 |

TABLE 9

| | | Score | |
|---|---|---|---|
| Stage | Indicator | E | G |
| Initial stage after | Spreadability | 0.9 | 0.1 |
| application | | | |
| Middle stage | Silkiness | 0.5 | 0.5 |
| after application | | | |
| Late stage after | Silkiness | 0 | 1 |
| application | Stickiness | 0.7 | 0.3 |
| | Moisture | 0.9 | 0.1 |

1-20. (canceled)

21. An oily composition comprising:
a) a first volatile acyclic silicone oil selected from among acyclic silicone oils with 2 to 4 silicon atoms, optionally containing C1-C6 alkyl groups, and mixtures thereof;
b) a second volatile acyclic silicone oil selected from among acyclic silicone oils with 5 to 6 silicon atoms, optionally containing C1-C6 alkyl groups, and mixtures thereof; and
c), a volatile hydrocarbon-based oil;
wherein ingredient (a) is present in an amount of from 10% to 40% based on the total weight of the composition;
ingredient (b) is present in an amount of from 20% to 40% based on the total weight of the composition; and
ingredient (c) is present in an amount of from 20% to 70% based on the total weight of the composition.

22. The oily composition of claim 1 further comprising:
a) a first volatile acyclic silicone oil selected from among acyclic silicone oils with 2 to 4 silicon atoms, optionally containing C1-C2 alkyl groups, and mixtures thereof;
b) a second volatile acyclic silicone oil selected from among acyclic silicone oils with 5 to 6 silicon atoms, optionally containing C1-C2 alkyl groups, and mixtures thereof; and
c), a volatile hydrocarbon-based oil;
wherein ingredient (a) is present in an amount of from 10% to 40% based on the total weight of the composition;
ingredient (b) is present in an amount of from 20% to 40% based on the total weight of the composition; and
ingredient (c) is present in an amount of from 20% to 70% based on the total weight of the composition.

23. The oily composition of claim 1, characterized in that ingredient (c) is selected from among branched alkanes containing 8 to 16 carbon atoms, or mixtures thereof.

24. The oily composition of claim 1, characterized in that ingredient (a) has a kinematic viscosity of from 0.65 to 1.5 cSt at 25° C.

25. The oily composition of claim 1, characterized in that ingredient (b) has a kinematic viscosity at 25° C. of greater than 1.5 cSt and less than 3 cSt.

26. The oily composition of claim 1, characterized in that ingredient (a) has a flash point of greater than or equal to −6° C. and less than 70° C.

27. The oily composition of claim 1, characterized in that ingredient (b) has a flash point of greater than or equal to 70° C. and less than 100° C.

28. The oily composition of claim 1, characterized in that ingredient (c) has a flash point of 40 to 70° C.

29. The oily composition of claim 1 further comprising:
a) octamethyltrisiloxane,
b) dodecamethylpentasiloxane, and
c) isododecane;
wherein octamethyltrisiloxane, dodecamethylpentasiloxane and isododecane, in terms of their contents, have the relationship: isododecane>dodecamethylpentasiloxane>octamethyltrisiloxane.

30. The oily composition of claim 1 further comprising:
a) hexamethyldisiloxane,
b) dodecamethylpentasiloxane, and
c) isododecane;
wherein hexamethyldisiloxane, dodecamethylpentasiloxane and isododecane, in terms of their contents, have the relationship: isododecane>dodecamethylpentasiloxane>hexamethyldisiloxane.

31. The oily composition of claim 1 further comprising:
a) decamethyltetrasiloxane,
b) dodecamethylpentasiloxane, and
c) isododecane;
wherein decamethyltetrasiloxane, dodecamethylpentasiloxane and isododecane, in terms of their contents, have the relationship: decamethyltetrasiloxane>isododecane≥dodecamethylpentasiloxane.

32. The oily composition of claim 1, characterized in that acyclic silicone oils with more than 7 silicon atoms are present in the oily composition in an amount of less than 1 wt %, based on the total weight of the oily composition.

33. The oily composition of claim 1, characterized in that cyclic silicone oils are present in the oily composition in an amount of less than 1 wt %, based on the total weight of the oily composition.

34. The oily composition of claim 1, characterized in that water is present in the oily composition in an amount of less than 1 wt %, based on the total weight of the oily composition.

35. The oily composition of claim 1, characterized in that powders are present in the oily composition in an amount of less than 1 wt %, based on the total weight of the oily composition.

36. The oily composition of claim 1, characterized in that plant-derived ingredients are present in the oily composition in an amount of less than 1 wt %, based on the total weight of the oily composition.

37. The oily composition of claim 1, characterized in that non-volatile oils are present in the oily composition in an amount of less than 1 wt %, based on the total weight of the oily composition.

38. The oily composition of claim 1, characterized in that ingredients (a), (b) and (c) are used in an amount of more than 99 wt %, based on the total weight of the oily composition.

39. Use of the oily composition of claim 1 in personal care and cosmetic products.

40. Use of the oily composition of claim 1 as a carrier liquid for siloxane polymers.
